(19) United States
(12) Patent Application Publication
(10) Pub. No.: US 2025/0355029 A1
(43) Pub. Date: Nov. 20, 2025

(54) METHOD AND SYSTEM FOR MEASURING RESPIRATION

(71) Applicant: SB Solutions Inc., Ulju-gun Ulsan (KR)

(72) Inventor: Young Mi HU, Ulsan (KR)

(73) Assignee: SB Solutions Inc., Ulju-gun Ulsan (KR)

(21) Appl. No.: 19/284,278

(22) Filed: Jul. 29, 2025

Related U.S. Application Data
(63) Continuation of application No. PCT/KR2023/013591, filed on Nov. 9, 2023.

(30) Foreign Application Priority Data
Jan. 31, 2023 (KR) 1020230012346

(51) Int. Cl. G01R 29/08; A61B 5/00

(52) U.S. Cl. CPC G01R 29/0814; A61B 5/4815; A61B 5/4818

(57) ABSTRACT

Disclosed is a method and system for measuring respiration. A respiration measurement method may include continuously measuring the change in the fringing field formed through a sensor attached to a subject according to respiration activity of the subject based on the change in a resonant frequency generated through an oscillator or iterative charging and discharging of the sensor, and providing information on the continuously measured change to determine information on respiration of subject through the continuously measured change.

### CROSS-REFERENCE TO RELATED APPLICATION(S)

This U.S. non-provisional application is a continuation application of PCT International Application PCT/KR2023/013591, which has an International filing date of Sep. 11, 2023, and claims priority under 35 U.S.C. 119 to Korean Patent Application No. 10-2023-0012346, filed on Jan. 31, 2023, in the Korean intellectual property office, the disclosures of which are herein incorporated by reference in its entirety.

### BACKGROUND

### 1. Field of the Invention

Example embodiments of the following description relate to a method and system for measuring respiration.

### 2. Description of the Related Art

With the increasing interest in health, research on a healthcare section using an electronic device is being actively conducted. For example, sensors mounted to the electronic device may collect information related to the electronic device, the outside of the electronic device, or a user, and it is important to continuously measure biosignals for the user to check the user's own condition. In this regard, as technology is required to be capable of monitoring the user's exercise status or abnormal condition, electronic devices that provide a function of checking the user's biosignals are being developed.

Related material includes Korean Patent Registration No. 10-2229999.

### SUMMARY

Example embodiments provide a respiration measurement method and device that may continuously measure respiration of a subject by continuously measuring the change according to respiration activity of the subject using a sensor attached to the subject.

According to an example embodiment, there is provided a respiration measurement method including continuously measuring the change in the fringing field formed through a sensor attached to a subject according to respiration activity of the subject based on the change in a resonant frequency generated through an oscillator or iterative charging and discharging of the sensor; and providing information on the continuously measured change to determine information on respiration of subject through the continuously measured change.

According to an aspect, the sensor may include at least two electrodes horizontally separate relative to the surface of the subject, and the continuously measuring may include forming the fringing field by applying a voltage to the at least two electrodes.

According to another aspect, the continuously measuring may include measuring the change in the resonant frequency of the oscillator as the fringing field changes according to the respiration activity of the subject.

According to still another aspect, the measuring the change in the resonant frequency of the oscillator may include counting a cycle of an output signal of the oscillator using a clock counter, and measuring the change in a counted value.

According to still another aspect, the clock counter may count the cycle of the output signal during a reference time generated by a reference time generator, and the higher the frequency of the output signal, the more cycles may be relatively counted by the clock counter during the reference time.

According to still another aspect, the continuously measuring may include iteratively charging and discharging the sensor; and measuring the change in the degree to which the sensor is charged as the fringing field changes according to the respiration activity of the subject.

According to still another aspect, the charging and the discharging may include charging and discharging the sensor at a reference time interval by connecting and disconnecting the sensor to and from a current source through a charge switch using a control signal of the reference time interval generated by a reference time generator.

According to still another aspect, the measuring the change in the degree to which the sensor is charged may include converting an input voltage of the sensor to a digital code using an analog-to-digital converter (ADC); and measuring the change in the degree to which the sensor is charged through a change in an output value of the ADC at a point in time at which charging of the sensor is terminated.

According to still another aspect, when it is determined that the respiration of the subject has not continued for a preset period of time or more based on information on the respiration of the subject, a notification may be provided.

According to an example embodiment, there is provided a respiration measurement system including a sensor configured to attach to a subject and to generate the fringing field; a measurement circuit configured to continuously measure the change in the fringing field according to respiration activity of the subject based on the change in a resonant frequency generated through an oscillator or iterative charging and discharging of the sensor; and a controller configured to control the measurement circuit and to provide information on the continuously measured change to determine information on the respiration of subject through the continuously measured change.

According to some example embodiments, it is possible to continuously measure respiration of a subject by continuously measuring the change according to respiration activity of the subject using a sensor attached to the subject.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates an example of a change in a sensor according to respiration activity of a subject according to an example embodiment;

FIG. 2 is a diagram illustrating an example of an internal configuration of a respiration measurement system according to an example embodiment;

FIG. 3 is a flowchart illustrating an example of a respiration measurement method according to an example embodiment;

FIG. 4 illustrates an example of a fringing field according to an example embodiment;

FIG. 5 illustrates an example of a measurement circuit unit according to an example embodiment;

FIG. 6 illustrates an example of an operation of a clock counter according to an example embodiment;

FIG. 7 illustrates another example of a measurement circuit unit according to an example embodiment; and

FIG. 8 illustrates an example of an operation of an analog-to-digital converter (ADC) according to an example embodiment.

### DETAILED DESCRIPTION

Hereinafter, example embodiments will be described in detail with reference to the accompanying drawings. However, various modifications may be made to the example embodiments, so the scope of the claims is not limited to or restricted by the example embodiments. It should be understood that all alterations and modifications to the example embodiments, equivalents, and substitutions thereof are included in the scope of the claims.

The terms used herein are for the purpose of describing particular example embodiments only and are not to be limiting of the example embodiments. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

Also, when describing the example embodiments with reference to the accompanying drawings, like reference numerals refer to like components and a repeated description related thereto will be omitted. When it is determined that detailed description of the related known art may unnecessarily obscure the gist of the example embodiments in describing the example embodiments, the detailed description is omitted.

In addition, terms such as first, second, A, B, (a), (b), and the like may be used herein to describe components of the example embodiments. Each of these terminologies is not used to define an essence, order, or sequence of a corresponding component, but used merely to distinguish the corresponding component from other component(s). When it is mentioned that one component is “connected” or “accessed” to another component, it may be understood that the one component is directly connected or accessed to another component or that still other component is “connected” or “accessed” between the two components.

Components included in one example embodiment and components including common functions will be described using the same names in other example embodiments. Unless otherwise stated, description made in one example embodiment may be applied to other example embodiments and specific description is omitted to the extent of overlap.

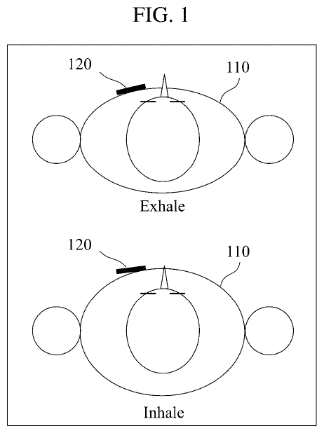

FIG. 1 illustrates an example of a change in a sensor according to respiration activity of a subject according to an example embodiment. FIG. 1 illustrates an example of a sensor 120 attached to a subject 110, such as a human or an animal that performs respiration activity. A part of the subject 110, such as the thorax, moves as its volume changes according to the respiration activity of the subject 110.

The sensor 120 may be attached to a specific part of the subject 110. Here, the sensor 120 may be attached to not be in complete contact with the outer surface of the subject 110. For example, in the case of a human body, the sensor 120 may be attached such that only the entire corresponding surface of the sensor 120 is not in close contact with the human skin, by allowing only a portion of one surface of the sensor 120 to be attached to the human skin.

When the subject 110 performs respiration activity, a movement occurs as the volume of the thoracic cage changes and the degree of adhesion between the sensor 120 and the outer surface of the subject 110 continuously changes according to this movement, which leads to inducing a certain change. FIG. 1 shows that the degree of adhesion between the sensor 120 and the subject 110 changes when the subject 110 inhales and exhales.

A respiration measurement system according to example embodiments may measure information on respiration, such as a respiration pattern and/or a respiration cycle of the subject 110 by continuously measuring the change according to the respiration activity of the subject 110.

In an example embodiment, the respiration measurement system may form the fringing field that is introduced into the surface of the subject 110 using two or more electrodes through the sensor 120. Depending on example embodiments, the fringing field may be formed to reach at least the surface of the subject 110. Here, the respiration measurement system may acquire information on the respiration of the subject 110 by measuring the change in the fringing field according to the respiration activity of the subject 110. Here, iterative charging and discharging of an oscillator and/or the sensor 120 may be utilized as a method of measuring the change in the fringing field according to the respiration activity of the subject 110.

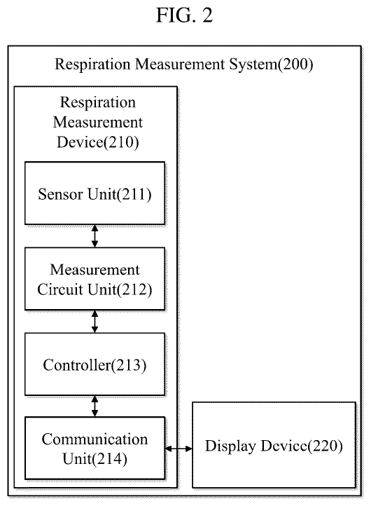

FIG. 2 is a diagram illustrating an example of an internal configuration of a respiration measurement system according to an example embodiment. A respiration measurement system 200 according to the example embodiment of FIG. 2 may be generally used to check normal respiration, that is, normal breathing during sleep and to diagnose sleep apnea, and may also be used to determine respiration quality and sleep quality, but is not limited thereto.

The respiration measurement system 200 may include a respiration measurement device 210 and a display device 220. An example embodiment of FIG. 2 describes an example in which each of the respiration measurement device 210 and the display device 220 is implemented as a separate physical device, but the respiration measurement device 210 and the display device 220 may be implemented as a single physical device depending on example embodiments.

This respiration measurement device 210 may include a sensor unit 211, a measurement circuit unit 212, a controller 213, and a communication unit 214.

The sensor unit 211 may be a respiration measurement sensor based on the change in the fringing field, and the measurement circuit unit 212 may include a measurement circuit configured to read sensor data (or sensing data) through the sensor unit 211. The controller 213 may control an operation of the measurement circuit unit 212, and may control the communication unit 214 to transmit the measured data to the display device 220. The communication unit 214 may include a communication module for wired/wireless connection with the display device 220. Data communication between the communication unit 214 and the display device 220 may be performed using at least one of various known communication protocols, such as Bluetooth Low Energy (BLE), near field communication (NFC), and WiFi.

The display device 220 may be a terminal of the user, such as a smartphone and a smart watch. The display device 220 may display data (e.g., waveform data) measured by the respiration measurement device 210 and may display the respiration rate, the respiration quality, and the sleep quality of the subject 110 determined through the measured data. To this end, an algorithm to determine the respiration rate, the respiration quality, and the sleep quality may run on the respiration measurement device 210 or the display device 220. When the corresponding algorithm runs on the respiration measurement device 210, the respiration measurement device 210 may further transmit, to the display device 220, information on the respiration rate, the respiration quality, and the sleep quality of the subject 110 determined using the measured data, in addition to the measured data.

Depending on example embodiments, an additional sensor, such as an exercise sensor, may be included in the respiration measurement device 210 to determine the sleep quality.

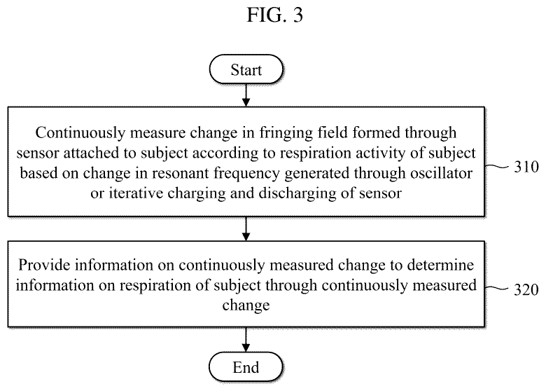

FIG. 3 is a flowchart illustrating an example of a respiration measurement method according to an example embodiment. The respiration measurement method according to the example embodiment may be performed by the fringing field-based respiration measurement device 210. In an example embodiment, the controller 213 of the respiration measurement device 210 may include at least one processor and a memory. Here, the operation of the respiration measurement device 210 may be interpreted to be implemented in such a manner that the processor of the controller 213 controls the measurement circuit unit 212 and the communication unit 214 included in the respiration measurement device 210 according to a code of a computer program stored in the memory of the controller 213.

In operation 310, the respiration measurement device 210 may continuously measure the change in the fringing field formed through a sensor attached to a subject according to respiration activity of the subject based on the change in a resonant frequency generated through an oscillator or iterative charging and discharging of the sensor. Here, the sensor attached to the subject may correspond to the sensor 120 of FIG. 1 or the sensor unit 211 of FIG. 2.

The sensor may include at least two electrodes horizontally separate relative to the surface of the subject. In this case, the respiration measurement device 210 may form the fringing field by applying a voltage to the at least two electrodes in operation 310. The fringing field may be formed to be introduced into the surface of the subject, or may be formed to at least reach the surface of the subject. Then, the respiration measurement device 210 may measure the change in the fringing field based on iterative charging and discharging of the oscillator or the sensor.

In an example embodiment, the respiration measurement device 210 may measure the change in the resonant frequency of the oscillator as the fringing field changes according to the respiration activity of the subject. For example, the fringing field may be formed inside the subject or on the surface of the subject. Here, to measure the change in the fringing field according to the respiration activity of the subject through the change in the resonant frequency of the oscillator, the respiration measurement device 210 may count a cycle of an output signal of the oscillator using a clock counter and may measure the change in a counted value.

Here, the clock counter may count the cycle of the output signal during a reference time generated by a reference time generator. The higher a frequency of the output signal, the more cycles may be relatively counted by the clock counter during the reference time.

That is, the change in the resonant frequency generated by the oscillator may be identified through the change in the value counted by the clock counter, which may represent that the change in the fringing field according to the respiration activity of the subject may be identified. As such, information on the respiration of the subject may be acquired by continuously measuring the change in the value counted by the clock counter.

In another example embodiment, the respiration measurement device 210 may iteratively charge and discharge the sensor attached to the subject. For example, the measurement circuit unit 212 included in the respiration measurement device 210 may charge and discharge the sensor at a reference time interval by connecting and disconnecting the sensor (e.g., capacitive sensor) to and from a current source through a charge switch using a control signal of the reference time interval generated through the reference time generator. Then, the respiration measurement device 210 may measure the change in the degree to which the sensor is charged according to the change in the fringing field according to the respiration activity of the subject. For example, the respiration measurement device 210 may have the fringing field that changes according to the change in the respiration activity of the subject and the change in electrostatic capacitance. Here, the change in the electrostatic capacitance may be measured through the change in the degree to which the sensor is charged. In this case, the measurement circuit unit 212 included in the respiration measurement device 210 may convert an input voltage of the sensor to a digital code using an analog-to-digital converter (ADC). Here, the measurement circuit unit 212 may measure the change in the degree to which the sensor is charged through a change in an output value of the ADC at a point in time at which charging of the sensor is terminated.

That is, the degree to which the sensor is charged may reflect the change in the fringing field according to the respiration activity of the subject, and the measurement circuit unit 212 may continuously measure the change in the output value at a point in time at which charging of the sensor is terminated (e.g., point in time at which the charge switch releases connection between the sensor and the current source) every time the sensor is charged and discharged. Therefore, information on the respiration of the subject may be acquired through the change in the output value of the ADC.

In operation 320, the respiration measurement device 210 may provide information on the continuously measured change to determine information on the respiration of the subject through the continuously measured change (change in fringing field). Information on the respiration of the subject may include information on the respiration rate, the respiration quality, and the sleep quality described above.

In an example embodiment, when the respiration measurement device 210 determines such information on the respiration, the respiration measurement device 210 may provide information on the continuously measured change (change in fringing field) as input to an algorithm that runs by the controller 213. The change measured substantially continuously may correspond to the change in the resonant frequency generated through the oscillator, and the change in the resonant frequency may be acquired through the change in the value counted by the clock counter, as described above.

In another example embodiment, when an external device of the respiration measurement device 210, such as the display device 220, determines information on the respiration, the respiration measurement device 210 may provide information on the continuously measured change to the external device such as the display device 220 through the communication unit 214.

Also, when it is determined that the respiration of the subject has not continued for a preset period of time or more based on information on the respiration of the subject, a notification may be provided. For example, when the respiration measurement device 210 directly determines information on the respiration of the subject, the respiration measurement device 210 may monitor whether the respiration of the subject has not continued for a preset period of time or more based on information on the respiration of the subject. In this case, the respiration measurement device 210 may provide a notification to the user using various methods, such as vibration and sound. As another example, the respiration measurement device 210 may transmit, to the display device 220, a signal to cause the display device 220 to provide the notification to the user using various methods, such as vibration and sound. As another example, when the display device 220 directly determines information on the respiration of the subject, the display device 220 may monitor whether the respiration of the subject has not continued for a preset period of time or more based on information on the respiration of the subject. In this case, the display device 220 may provide the notification to the user using various methods such as vibration and sound. As another example, the display device 220 may transmit, to the respiration measurement device 210, a signal to cause the respiration measurement device 210 to provide the notification to the user using various methods such as vibration and sound. Here, the user may be the subject, a guardian of the subject, and/or an administrator of the subject. Also, the preset period of time may be empirically determined, for example, 8 seconds and 10 seconds.

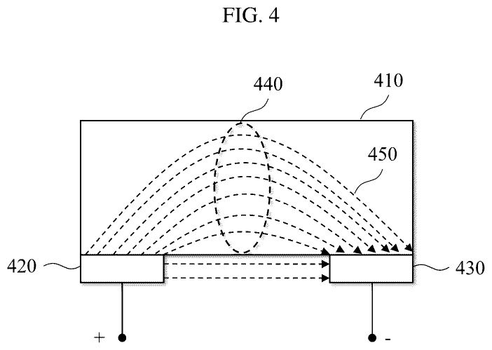

FIG. 4 illustrates an example of a fringing field according to an example embodiment. FIG. 4 shows two electrodes 420 and 430 attached to an MUT 410. Here, in response to a voltage being applied to the two electrodes 420 and 430, the fringing field 440 may be formed inside the MUT 410 between the two electrodes 420 and 430 as shown in FIG. 4.

In FIG. 4, the fringing field 440 is indicated by a dotted ellipse to help understanding. However, in reality, the fringing field 440 may be formed by electromagnetic force lines (e.g., field lines 450 of FIG. 4) when biasing a voltage on a capacitor.

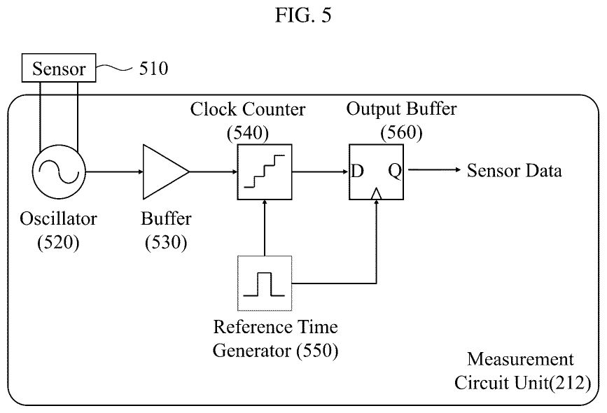

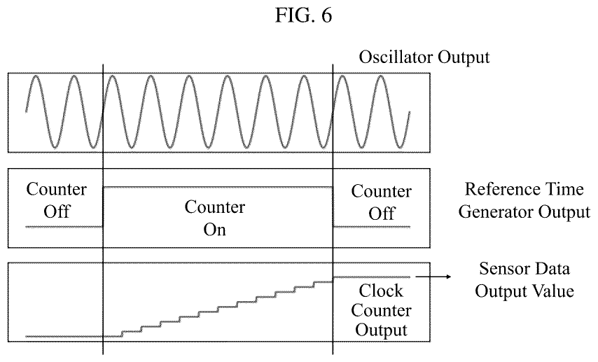

FIG. 5 illustrates an example of a measurement circuit unit according to an example embodiment, and FIG. 6 illustrates an example of an operation of a clock counter according to an example embodiment.

The measurement circuit unit 212 according to the example embodiment of FIG. 5 may include an oscillator 520 connected to a sensor 510, a buffer 530, a clock counter 540, a reference time generator 550, and an output buffer 560.

The sensor 510 may correspond to the sensor 120 or the sensor unit 212 described above, and, in response to a voltage being applied to at least two electrodes (e.g., two electrodes 420 and 430) included in the sensor 510, the fringing field may be formed. The oscillator 520 may be a resistor-capacitor (RC) oscillator or an inductor-capacitor (LC) oscillator. Here, if the fringing field varies according to respiration, an output frequency (resonant frequency) of the oscillator 520 connected to the sensor 510 may vary. In this case, an output signal of the oscillator 520 may be input to the clock counter 540 through the buffer 530.

The clock counter 540 may count a cycle of an input signal during a reference time of the reference time generator 550. The higher the frequency of the input signal, the more cycles may be relatively counted during the reference time, so an output value of the clock counter 540 may increase. The reference time generator 550 may generate a signal of the reference time in which the clock counter 540 operates.

The output of the clock counter 540 may be output as sensor data through the output buffer 560.

FIG. 6 shows an example in which, when output (signal of resonant frequency) of the oscillator 520 is input to the clock counter 540, the clock counter 540 counts a cycle of output of the oscillator 520 according to output of the reference time generator 550 and outputs the same as an output value of sensor data.

In this manner, the fringing field formed through the sensor 510 may change according to the respiration of the subject 110, the resonant frequency output from the oscillator 520 may change in response to the change in the fringing field, and the output value of the clock counter 540 may change according to the change in the resonant frequency. Therefore, conversely, information on the respiration of the subject 110 may be acquired through the change in the output value of the clock counter 540.

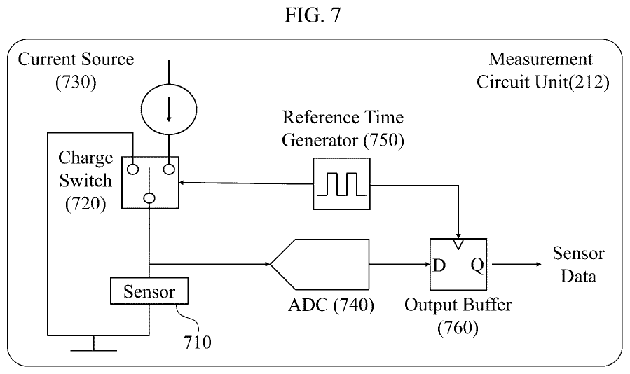

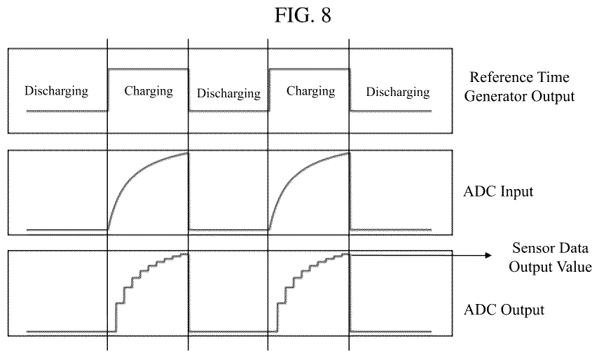

FIG. 7 illustrates another example of a measurement circuit unit according to an example embodiment, and FIG. 8 illustrates an example of an operation of an ADC according to an example embodiment.

The measurement circuit unit 212 according to the example embodiment of FIG. 7 may include a charge switch 720 connected to a sensor 710, a current source 730, an ADC 740, a reference time generator 750, and an output buffer 760.

The sensor 710 may correspond to the sensor 120 or the sensor unit 212 described above. In the example embodiment of FIG. 7, the sensor 710 is illustrated as being included in the measurement circuit unit 212, but in reality, the sensor 710 may be provided outside the measurement circuit unit 212 to be attached to the subject 110.

The measurement circuit unit 212 may measure the degree of charge by iteratively charging and discharging the sensor 710 using the charge switch 720. The reference time generator 750 may generate a control signal of a reference time interval and may operate the charge switch 720. If the charge switch 720 is turned on, the sensor 710 and the current source 730 may be connected and the sensor 710 may be charged accordingly, and if the charge switch 720 is turned off, connection between the sensor 710 and the current source 730 may be disconnected and the sensor 710 may be discharged accordingly.

While the sensor 710 is being charged, an input voltage of the sensor 710 may increase and the measurement circuit unit 212 may convert this voltage to a digital code using the ADC 740. Here, an output value of the ADC 740 at a point in time at which the reference time generator 750 turns off the charge switch 720 may be output as sensor data through the output buffer 760.

FIG. 8 illustrates input and output of the ADC 740 as the charge switch 720 repeats connection and disconnection between the sensor 710 and the current source 730 according to output of the reference time generator 750. Also, it is shown that an output value of the ADC 740 at a point in time at which the reference time generator 750 turns off the charge switch 720 may be output as an output value of sensor data.

As described above, as the fringing field changes according to the respiration of the subject 110, the degree to which the sensor 710 is charged may change and the output value of the ADC 740 may change according to the change in the degree to which the sensor 710 is charged. Therefore, conversely, information on the respiration of the subject 110 may be acquired through the change in the output value of the ADC 740.

As described above, according to example embodiments, it is possible to continuously measure respiration of a subject by continuously measuring a change according to respiration activity of the subject using a sensor attached to the subject.

The systems or the apparatuses described herein may be implemented using hardware components, software components, and/or a combination of the hardware components and the software components. For example, the apparatuses and the components described herein may be implemented using one or more general-purpose or special purpose computers, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that the processing device may include multiple processing elements and/or multiple types of processing elements. For example, the processing device may include multiple processors or a processor and a controller. In addition, other processing configurations are possible, such as parallel processors.

The software may include a computer program, a piece of code, an instruction, or some combinations thereof, for independently or collectively instructing or configuring the processing device to operate as desired. Software and/or data may be embodied in any type of machine, component, physical equipment, virtual equipment, computer storage medium or device, to provide instructions or data to the processing device or be interpreted by the processing device. The software also may be distributed over network coupled computer systems so that the software is stored and executed in a distributed fashion. The software and data may be stored by one or more computer readable storage mediums.

The methods according to various example embodiments may be implemented in a form of a program instruction executable through various computer methods and recorded in computer-readable media. The computer-readable media may include, alone or in combination with program instructions, data files and data structures. Here, the media may be to continuously store a computer-executable program or to temporarily store the same for execution or download. The media may be various types of recording methods or storage methods in which a single piece of hardware or a plurality of pieces of hardware are combined and may be distributed over a network without being limited to a medium that is directly connected to a computer system. Examples of the media include magnetic media such as hard disks, floppy disks, and magnetic tapes; optical media such as CD ROM and DVD; magneto-optical media such as floptical disks; and hardware devices that are specially configured to store and perform program instructions, such as read-only memory (ROM), random access memory (RAM), flash memory, and the like. Examples of other media may include recording media and storage media managed by an app store that distributes applications or a site, a server, and the like that supplies and distributes other various types of software. Examples of the program instructions include an advanced language code executable by a computer using an interpreter, as well as a machine language code as produced by a compiler.

Although the example embodiments are described with reference to some specific example embodiments and accompanying drawings, it will be apparent to one of ordinary skill in the art that various alterations and modifications in form and details may be made in these example embodiments without departing from the spirit and scope of the claims and their equivalents. For example, suitable results may be achieved if the described techniques are performed in different order, and/or if components in a described system, architecture, device, or circuit are combined in a different manner, and/or replaced or supplemented by other components or their equivalents.

Therefore, other implementations, other example embodiments, and equivalents of the claims are to be construed as being included in the claims.

What is claimed is:

1. A respiration measurement method comprising:
continuously measuring the change in the fringing field formed through a sensor attached to a subject according to respiration activity of the subject based on the change in a resonant frequency generated through an oscillator or iterative charging and discharging of the sensor; and
providing information on the continuously measured change to determine information on respiration of the subject through the continuously measured change,
wherein the continuously measuring comprises:
iteratively charging and discharging the sensor; and
measuring the change in the degree to which the sensor is charged as the fringing field changes according to the respiration activity of the subject.

2. The respiration measurement method of claim 1, wherein the sensor includes at least two electrodes horizontally separate relative to the surface of the subject, and
the continuously measuring comprises forming the fringing field by applying a voltage to the at least two electrodes.

3. The respiration measurement method of claim 1, wherein the continuously measuring comprises measuring the change in the resonant frequency of the oscillator as the fringing field changes according to the respiration activity of the subject.

4. The respiration measurement method of claim 3, wherein the measuring the change in the resonant frequency of the oscillator comprises counting a cycle of an output signal of the oscillator using a clock counter, and measuring the change in a counted value.

5. The respiration measurement method of claim 4, wherein the clock counter counts the cycle of the output signal during a reference time generated by a reference time generator, and
the higher a frequency of the output signal, the more cycles are relatively counted by the clock counter during the reference time.

6. The respiration measurement method of claim 1, wherein the charging and the discharging comprises charging and discharging the sensor at a reference time interval by connecting and disconnecting the sensor to and from a current source through a charge switch using a control signal of the reference time interval generated by a reference time generator.

7. The respiration measurement method of claim 1, wherein the measuring the change in the degree to which the sensor is charged comprises:
converting an input voltage of the sensor to a digital code using an analog-to-digital converter (ADC); and
measuring the change in the degree to which the sensor is charged through a change in an output value of the ADC at a point in time at which charging of the sensor is terminated.

8. The respiration measurement method of claim 1, wherein, when it is determined that the respiration of the subject has not continued for a preset period of time or more based on information on the respiration of the subject, a notification is provided.

9. A respiration measurement system comprising:
a sensor configured to attach to a subject and to generate the fringing field;
a measurement circuit configured to continuously measure the change in the fringing field according to respiration activity of the subject based on the change in a resonant frequency generated through an oscillator or iterative charging and discharging of the sensor; and
a controller configured to control the measurement circuit and to provide information on the continuously measured change to determine information on respiration of subject through the continuously measured change,
wherein the measurement circuit is configured to iteratively charge and discharge the sensor, and to measure the change in the degree to which the sensor is charged as the fringing field changes according to the respiration activity of the subject.
